(19) United States
(12) Patent Application Publication
(10) Pub. No.: US 2024/0091513 A1
(43) Pub. Date: Mar. 21, 2024

(54) Vacuum-Assisted Treatment Device, and Related Systems and Methods

(71) Applicant: Inovio Pharmaceuticals, Inc., Plymouth Meeting, PA (US)

(72) Inventors: Paul Fisher, Fallbrook, CA (US); Jay McCoy, Temecula, CA (US); Alison A. Generotti, Ocean City, NJ (US); Andrea Kemme, San Diego, CA (US)

(21) Appl. No.: 18/468,041

(22) Filed: Sep. 15, 2023

Related U.S. Application Data
(60) Provisional application No. 63/375,979, filed on Sep. 16, 2022.

(51) Int. Cl. A61M 37/00; A61N 1/04; A61N 1/32

(52) U.S. Cl. CPC A61M 37/00; A61N 1/0476; A61N 1/327; A61M 2037/0007; A61N 1/042; A61N 1/0424

(57) ABSTRACT

Techniques for treating subjects with an injected agent include applying vacuum pressure to tissue adjacent the injection and optionally electroporating said tissue. A device for applying such treatment includes a housing defining a receptacle and an opening into the receptacle. A port extends through the housing remote from the opening and is connectable to a vacuum source, such that the port can communicate vacuum pressure from the vacuum source to the receptacle. The device includes an insert that is receivable within the receptacle and that defines an internal chamber, which is in communication with the vacuum source when the insert resides in the receptacle. The insert includes at least one electrode positioned within the chamber and configured to deliver one or more electroporation pulses to a targeted portion of tissue extending through the at least one opening and at least momentarily held in the chamber responsive to the vacuum pressure.

### CROSS-REFERENCE TO RELATED APPLICATIONS

This application claims the benefit of U.S. Provisional Application No. 63/375,979, filed Sep. 16, 2022, the entire contents of which are incorporated herein by this reference.

The present application is related to U.S. Patent Publication No. 2021/0290941 A1, published Sep. 23, 2021, in the name of Fisher, et al. (“the '941 Reference”), the entire contents of which are incorporated by this reference.

### TECHNICAL FIELD

The present invention relates to devices, assemblies, and systems for gripping and deforming tissue with vacuum pressure, injecting fluid into the tissue, and optionally electroporating the tissue with electrodes.

### BACKGROUND

Plasmid DNA delivery via electroporation (EP) is a clinically proven vaccination platform with potential safety, immunogenicity, and cost advantages compared to traditional vectors. EP is the temporary permeabilization of cell membranes due to electric fields, which greatly enhances DNA uptake within the electroporated tissue. The design of an EP delivery system can directly impact the magnitude and kinetics of immune responses. Clinical EP devices typically use needle electrodes inserted into skin or muscle tissue at the site of DNA injection, and due to the reliability of this method, needle-based EP has become the standard for in vivo DNA delivery.

As an alternative to intramuscular EP (IM-EP), the skin is an attractive target for EP not only due to its accessibility, which permits shallower EP fields, smaller needles, and increased tolerability, but also due to its rich population of immune cells which may permit fractional dosing. Intradermal EP (ID-EP) using a minimally invasive needle array is more pain-tolerable than IM-EP, and is also capable of generating potent immune responses at a fraction of the IM dose.

Although needle electrodes are most commonly used in the clinic because they generate reliable electric fields throughout a predetermined tissue depth, the advent of ID-EP has brought new focus to minimally- or non-invasive EP alternatives. Most non-invasive EP devices function by contacting the skin surface with non-penetrating electrodes and conducting current through the highly resistive stratum corneum and the underlying viable epidermis, generating a shallow electric field at the site of the ID plasmid injection. A challenge associated with non-invasive ID-EP is the risk of the electrodes losing electrical contact during the procedure, which can result in tissue damage, incomplete treatment, and electrical arcing. To overcome this issue, most noninvasive electrode arrays require constant downward pressure, clamping mechanisms, and/or adhesives to prevent electrodes from dislodging or moving during EP. These requirements can be logistical barriers to adoption or can contribute to inconsistency and/or operator error that can compromise successful vaccine delivery. Furthermore, when the electrodes of noninvasive EP devices are placed flat against the skin, the resulting electric field is quite shallow due to the large voltage drop across highly resistive stratum corneum and the superficial path of current flow, resulting in primarily keratinocytes, Langerhans cells, and other superficial cell types being transfected. In contrast, needle-based EP devices additionally transfect deeper tissue layers with a more diverse cellular population, which can include dendritic cells, adipocytes, fibroblasts, and myocytes.

### BRIEF DESCRIPTION OF THE DRAWINGS

The foregoing summary, as well as the following detailed description of illustrative embodiments of the present application, will be better understood when read in conjunction with the appended drawings. For the purposes of illustrating the features of the present application, there is shown in the drawings illustrative embodiments. It should be understood, however, that the application is not limited to the precise arrangements and instrumentalities shown. In the drawings:

FIG. 1 is a diagrammatical view of an electroporation system that employs a vacuum-assisted electroporation cup (or “vacuum cup”) in combination with a needle injection device, according to an embodiment of the present disclosure;

FIG. 2A is a perspective view of the vacuum cup illustrated in FIG. 1, showing a vacuum chamber of the cup and an array of electrodes located within the chamber, according to an embodiment of the present disclosure;

FIG. 2B is a bottom plan view of the vacuum cup illustrated in FIG. 2A, showing a pattern in which the electrodes are arrayed;

FIG. 2C is a plan elevation view of one of the electrodes illustrated in FIGS. 2A-2B;

FIG. 2D is a sectional side view of the vacuum cup illustrated in FIG. 2A, taken along a central axis of the cup;

FIG. 2E is an enlarged sectional view of region 2E-2E illustrated in FIG. 2D;

FIG. 2F is an enlarged sectional view of region 2F-2F illustrated in FIG. 2D;

FIG. 3 is a sectional side view of kit that includes a vacuum cup and a plurality of interchangeable inserts having different chamber volumes, according to an embodiment of the present disclosure;

FIGS. 4A-4C are views of a virtual three-dimensional (3D) tissue model, generated using mesh modeling techniques, showing a volume of tissue as pulled into a vacuum cup, according to an embodiment of the present disclosure;

FIG. 4D shows charts depicting measured tissue heights within vacuum cup prototypes that were constructed using the models shown in FIGS. 4A-4C;

FIG. 4E is a graph showing a sigmoid equation used to model electric field-dependent increases in tissue conductivity for vacuum cups;

FIGS. 5A-5F show simulated electrical field strengths created in tissue according to various chamber diameters and applied voltages and vacuum pressures;

FIGS. 5G-5H are graphs comparing the delivered current as calculated using the simulations shown in FIGS. 5A-5F versus measured currents based on vacuum cup prototypes;

FIG. 6 is a diagram showing gene expression in guinea pig skin (on the external and internal skin surfaces) obtained following plasmid delivery of a plasmid encoding the gene for green fluorescent protein (GFP) and then treated at various vacuum pressures and electroporation voltages using a vacuum cup;

FIG. 7A is a graph showing 6-week humoral immunogenicity data in guinea pigs after treatments with a DNA vaccine against MERS, particularly comparing humoral immune responses following treatments in skin using mantoux injection followed by various applications of vacuum pressure and electroporation voltage; and

FIG. 7B is a chart showing cellular immune responses at week 4 from the same study illustrated in FIG. 7A;

### DETAILED DESCRIPTION OF ILLUSTRATIVE EMBODIMENTS

The present disclosure can be understood more readily by reference to the following detailed description taken in connection with the accompanying figures and examples, which form a part of this disclosure. It is to be understood that this disclosure is not limited to the specific devices, methods, applications, conditions or parameters described and/or shown herein, and that the terminology used herein is for the purpose of describing particular embodiments by way of example only and is not intended to be limiting of the scope of the present disclosure. Also, as used in the specification including the appended claims, the singular forms “a,” “an,” and “the” include the plural, and reference to a particular numerical value includes at least that particular value, unless the context clearly dictates otherwise.

The term “plurality,” as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

The terms “approximately,” “substantially,” and “about,” as used herein with respect to dimensions, angles, and other geometries, takes into account manufacturing tolerances. Further, the terms “approximately,” “substantially,” and “about” can include 10% greater than or less than the stated dimension or angle. Further, the terms “approximately,” “substantially,” and “about” can equally apply to the specific value stated.

The term “agent,” as used herein, means a polypeptide, a polynucleotide, a small molecule, or any combination thereof. The agent may be a recombinant nucleic acid sequence encoding an antibody, a fragment thereof, a variant thereof, or a combination thereof. The agent may be formulated in water or a buffer, such as saline-sodium citrate (SSC) or phosphate-buffered saline (PBS), by way of non-limiting examples.

The term “injectate,” as used herein, means an agent configured for injection in tissue.

The term “intradermal” (ID), as used herein, means within the layer of skin that includes the epidermis (i.e., the epidermal layer, from the stratum corneum to the stratum basale) and the dermis (i.e., the dermal layer).

The term “intramuscular” (IM), as used herein, means within muscle tissue.

The term “adipose,” as used herein, means the layer containing adipocytes (i.e., fat cells) that reside in the subcutaneous layer.

The term “electroporation,” as used herein, means employing an electrical field within tissue that temporarily and reversibly increases the permeability and/or porosity of the cell membranes of cells in the tissue, thereby allowing an agent, for example, to be introduced into the cells.

The term “electroporation field,” as used herein, means an electric field capable of electroporating cells. In instances where an electric field includes a portion that is capable of electroporating cells and another portion that is incapable of electroporating cells, the “electroporation field” refers specifically to that portion of the electric field that is capable of electroporating cells. Thus, an electroporation field can be a subset of an electric field.

The term “zone,” as used herein, means a volume of space, such as a volume of space within tissue.

The term “transfection zone,” as used herein, means a volume of tissue in which transfection occurs, and can be used synonymously with the term “transfection volume.”

The embodiments described below pertain to systems and devices that perform vacuum-assisted delivery of injectates to tissue, particularly a targeted layer of tissue, such as intradermal tissue and/or adipose tissue. These embodiments subject a targeted volume of the tissue (or “tissue volume”) to vacuum pressure (i.e., negative pressure) to deform the tissue in a manner favorable for redistribution of fluid, such as migration of an injected agent, within a target zone in the tissue layer. In particular, an open end of a vacuum device, such as a vacuum cup, is placed in contact with an outer surface of the tissue (e.g., the “skin”) overlying the tissue volume, and vacuum pressure is applied to an interior of the cup, thereby drawing the tissue volume into the vacuum cup, which positionally secures the tissue volume to the cup. This can allow, for example, electrodes positioned within the vacuum cup to generate a predictable, substantially uniform electroporation field within the tissue volume, thereby resulting in a predictable, substantially uniform transfection zone within the tissue volume. The vacuum pressure provided by the embodiments described below has also been observed to provide favorable redistribution of fluid within the tissue volume, including favorable in vivo dispersion of an injectate within the tissue volume, and also favorable in vivo ingress and egress of fluid into and out of the target zone. Stated differently, the vacuum pressure enhances the dispersion of the injectate throughout the tissue to enlarge the transfection zone and also draws more in vivo fluids into the target zone, increasing the amount of cells that are exposed to transfected cells. The inventors have observed that the vacuum-assisted electroporation treatments described throughout this disclosure have resulted in subjects' increased responses to injectates.

The inventors have also observed that application of vacuum pressure can cause transfection within the tissue volume even without electroporation, as more fully described in the '941 Reference.

Furthermore, the embodiments described below can also be adaptable between uses and/or during use without mechanical reconfiguration. For example, during and/or between uses, electrical parameters of the electrodes can be adjusted as needed to manipulate the electroporation field in the tissue volume to achieve favorable treatment results. Additionally or alternatively, the vacuum pressure can be adjusted as needed during and/or between uses to physically manipulate the tissue volume to achieve favorable treatment results. For example, a higher vacuum pressure can be applied to draw a larger tissue volume into vacuum cup and a lower vacuum pressure can be applied to draw a lesser tissue volume into the vacuum cup. In this manner, the same vacuum cup can be employed to target different tissue layers for electroporative treatment by selectively exposing different tissue layers to the electroporation field. Additionally, the vacuum pressure can be pulsed during use to manipulate the mechanical behavior of the targeted tissue, such as to enhance fluid redistribution within the tissue.

The embodiments described below can also be mechanically adaptable between uses and/or during use. For example, the vacuum cup can include an outer housing and an internal insert that defines the cup's internal volume for receiving tissue. The internal insert defines an internal cup diameter, and can be interchangeable with various other internal inserts having different internal diameters. In this manner, the outer housing can be coupled with different internal inserts that provide different internal volume characteristics for adapting the vacuum treatment, such between treatments and/or during a single treatment.

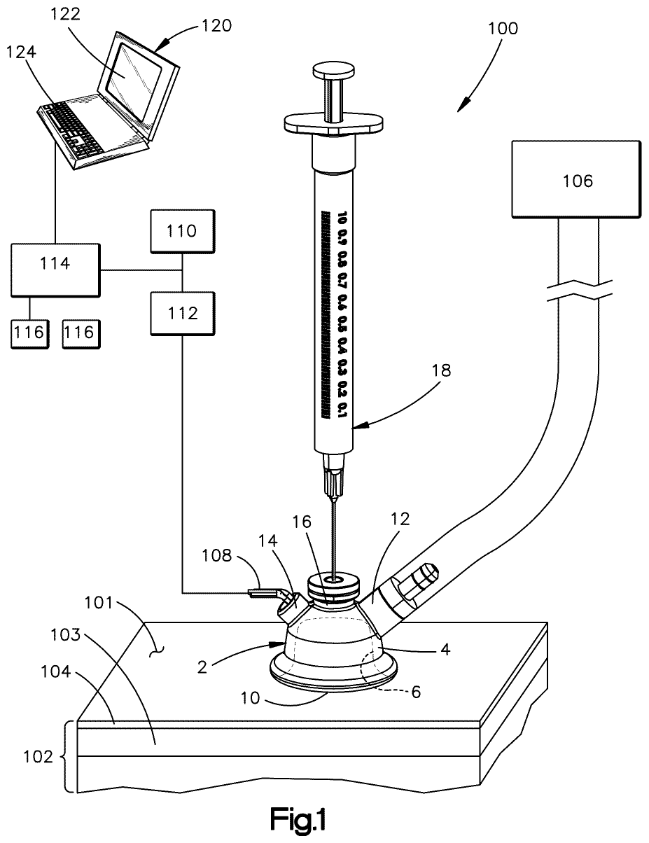

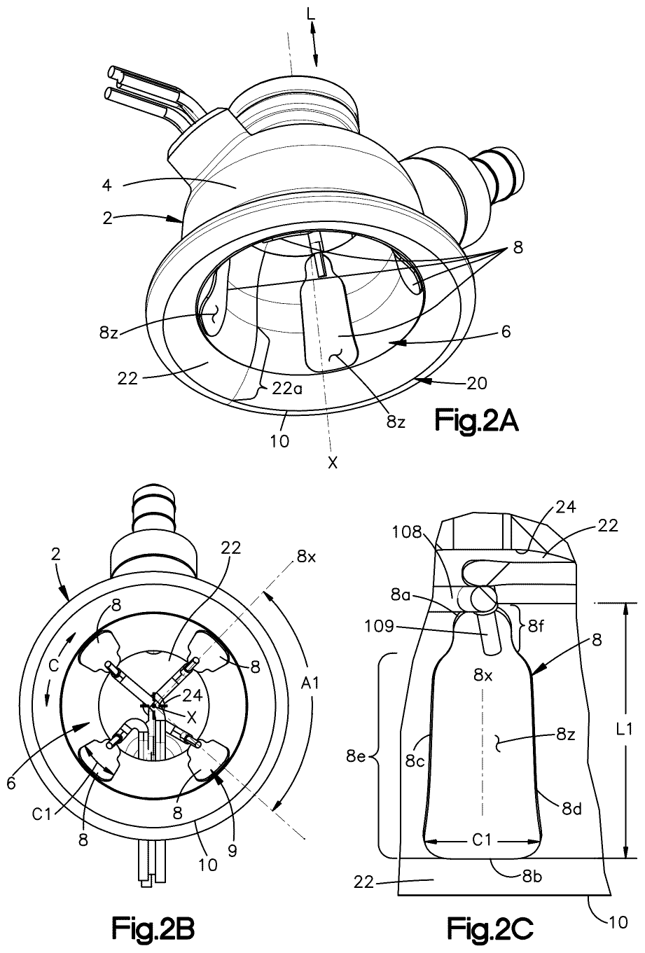

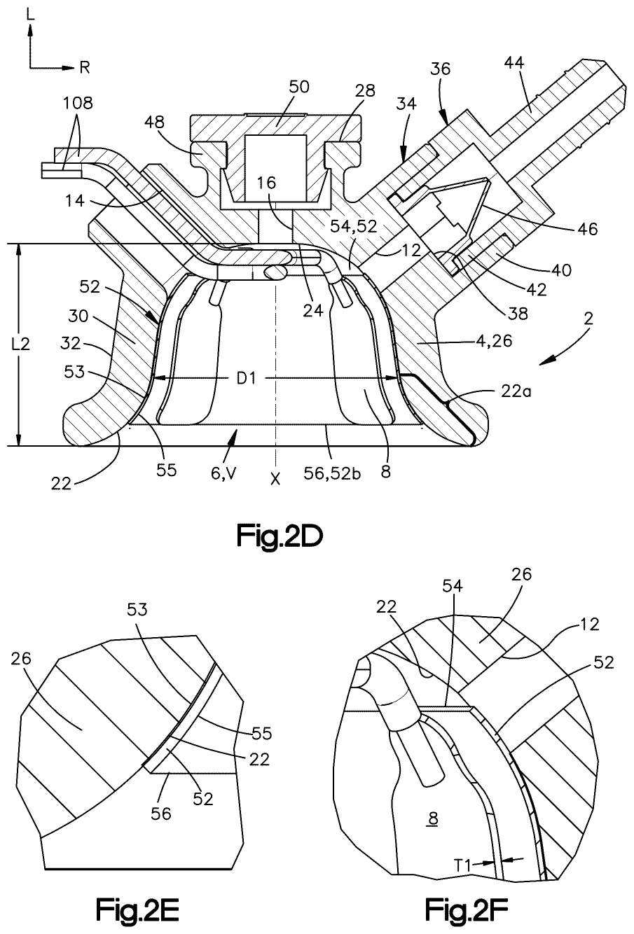

Referring now to FIG. 1, an example injection treatment system 100 for treating a patient according to the present disclosure includes a vacuum-assisted electroporation device 2, which includes a housing 4 that defines an internal vacuum chamber 6 and at least one electrode 8 (see, e.g., FIGS. 2A-2C) positioned within the chamber 6. The device 2 can also be referred to as a “vacuum cup” or simply a “cup.” The housing 4 can be referred to as a “cup housing”. The vacuum cup 2 is configured so that a physician can place a distal end 10 of the vacuum cup 2 onto an outer surface 101 of tissue 102 targeted for electroporation treatment and can apply vacuum pressure to the vacuum chamber 6 to draw, pull, or otherwise induct the tissue 102 into the vacuum chamber 6 and into contact with the electrode(s) 8 therein. The electrode(s) 8 are configured to deliver one or more electroporation pulses to the tissue 102 drawn into the chamber 6 and held therein by the vacuum pressure. The tissue 102 includes the layer targeted for treatment, such as adipose tissue 103 (also referred to herein as the “adipose layer” 103) or intradermal tissue 104 (also referred to herein as the “skin layer” 104).

In the illustrated examples, the device 2 includes a plurality of electrodes 8. It should be appreciated, however, that in other embodiments, the device 2 can include a single electrode 8. Thus, although the following description of the illustrated embodiments refers to plural electrodes 8, such disclosure also encompasses embodiments with a single electrode 8, unless otherwise stated. In embodiments with plural electrodes 8, the electrodes 8 are arranged into an array 9 of electrodes 8, which can also be referred to as an electrode array 9.

The vacuum cup 2 includes one or more couplings, such as ports, for connection to one or more external components. For example, the vacuum cup 2 has a first port 12 for providing fluid communication between the vacuum chamber 6 and a vacuum source 106, such as a vacuum pump. The vacuum cup 2 can also have a second port 14 for providing access to circuitry 108 providing electrical communication between the electrodes 8 and an energy source 110, such as a power generator. The vacuum cup 2 can further include an optional third port 16, such as for providing an external tool, such as an injection device 18 carrying an injectate with access to the vacuum chamber 6. As shown, the injection device 18 can be a hypodermic needle, although the vacuum cup 2 can be adapted for use with other types of injection devices 18, including jet injection devices, as described in more detail below. It should also be appreciated that the vacuum cup 2 can optionally be employed after the agent is injected into the tissue 102. In such embodiments, the vacuum cup 2 need not include the third port 16.

The energy source 110 can be in electrical communication with a signal generator 112, such as a waveform generator, for generating and transmitting an electric signal in the form of one or more electrical pulses having particular electrical parameters to the electrodes 8 for electroporating cells within the tissue 102 in the vacuum chamber 6. Such electrical parameters include electrical potential (voltage), electric current type (alternating current (AC) or direct current (DC)), electric current magnitude (amperage), pulse duration, pulse quantity (i.e., the number of pulses delivered), and time interval or “delay” between pulses (in multi-pulse deliveries). The signal generator 112 can include a waveform logger for recording the electrical parameters of the pulse(s) delivered. The signal generator 112 can be in electrical communication with a control unit 114 (also referred to herein as a “controller”), which can include a processor 116 configured to control operation of the electroporation system 100, including operation of the signal generator 112. The processor 116 can be in electronic communication with computer memory 118, and can be configured to execute software and/or firmware including one or more algorithms for controlling operation of the system 100. The processor 116 can be in electrical communication with a user interface 120, which can include a display 122 for presenting information relating to operation of the system 100 and a keypad 124 allowing an operator, such as physician, to input information, such as commands, relating to operation of the system 100. It should be appreciated that the display 122 can be a touchscreen display allowing the operator to input information directly at the display 122. It should also be appreciated that the interface 120 can be computer interface, such as a table-top computer or laptop computer, or a hand-held electronic device, such as a smart-phone or the like.

Referring now to FIGS. 2A-2B, the distal end 10 of the vacuum cup 2 defines at least one opening 20 leading into the vacuum chamber 6. The opening 20 can be circular as shown, although other opening shapes are within the scope of the present disclosure, as described in more detail below. The distal end 10 of the vacuum cup 2 (and thus also the opening 20) can be defined by the housing 4, which can define an interior surface 22 that extends from the distal end 10 of the housing to a proximal end 24 of the chamber 6. Accordingly, the chamber 6 also extends from the distal end 10 to the proximal end 24. The interior surface 22 at least partially defines the bounds of the vacuum chamber 6. The interior surface 22 preferably has a bell-shaped or “bell curve” geometry. A distal portion 22a of the interior surface 22 leading into the chamber 6 from the distal end 10 can have a tapered, radiused contour for reducing or otherwise mitigating damage, such as bruising, to the tissue at the periphery of the distal end 10 during use of the vacuum cup 2. The distal portion 22a can be referred to as a “lead-in” portion 22a of the interior surface 22. A proximal portion of the interior surface 22, such as at the proximal end 24 of the vacuum chamber 6, can be referred to as a “proximal end surface” or simply “end surface” of the vacuum chamber 6.

At least one and up to all of the electrodes 8 extend alongside the interior surface 22. As shown, the electrodes 8 can extend alongside the interior surface 22 between the distal end 10 and the proximal end 24 with respect to a longitudinal direction L oriented along a central axis X of the housing 4. The electrodes 8 of the present embodiment are preferably substantially rigid, although in other embodiments the electrodes 8 can have a measure of flexibility. The electrodes 8 can comprise thin layers of conductive material coupled to (e.g., via coating, deposition, bonding, and/or adhesion) associated substantially rigid, non-conductive support bodies, which can be constructed of plastics or other suitable non-conductive materials. The electrodes 8 can have surface geometries that are substantially conformal with the interior surface 22. The electrodes 8 can be elongate along a direction having a directional component along the longitudinal direction L. The electrodes 8 can also extend alongside the interior surface 22 along a circumferential direction C about the central axis X. As shown in FIGS. 2B-2C, the electrodes 8 can each define a circumferential dimension C1 (or “width” C1) measured along the circumferential direction C. The electrodes 8 can be positioned at regular angular intervals A1 about the central axis X. The angular intervals A1 can be measured from respective central axes 8x of the electrodes 8. As shown in FIG. 2B, the electrodes 8 can be positioned, for example, at ninety-degree angular intervals A1 about the central axis X. Thus, it can be said that the electrodes 8 are symmetrically spaced about the central axis X. It should be appreciated that other angular intervals A1 between electrodes 8 are within the scope of the present disclosure, as described in more detail below. Moreover, in some embodiments, the angular intervals A1 between electrodes 8 can vary along the interior surface 22. That is, the electrodes 8 can be spaced at irregular intervals about the central axis X. Furthermore, the electrodes 8 need not be symmetrically spaced about the central axis X.

As shown in FIG. 2C, each electrode 8 can define an electrode length L1 measured from a first end 8a to a second end 8b of the electrode 8 spaced from each other along the central electrode axis 8x. The electrodes 8 can also have first and second sides 8c, d spaced from each other to define an electrode width C1 along the circumferential direction C. Each electrode 8 can have an interior electrode surface 8z that is configured to contact the tissue surface 101 for delivering the one or more electroporation pulses. The interior electrode surface 8z can extend from the first end 8a to the second end 8b and from the first side 8c to the second side 8d of the electrode 8. Each electrode 8 can define a primary or “contact” portion 8e that extends from the second electrode end 8b towards the first electrode end 8a and also extends from the first to the second side 8a, b of the electrode 8. As shown, the electrode width C1 can be measured between the first and second electrode sides 8a, b, and need not be uniform along the contact portion 8e. The electrode length L1 and width C1 can each be in a range from about 1.0 mm to about 30 mm, more particularly in a range of about 2 mm to about 25 mm, and more particularly in a range of about 4 mm to about 20 mm. The electrodes 8 can define a thickness T1 (see FIG. 2F) in a range from about 0.0005 mm to about 2.000 mm. It should be appreciated that the electrode length L1 can be greater than, less than, or equivalent to the electrode width C1.

The portion of the internal electrode surface 8z within the contact portion 8e can be referred to as a “contact surface” 8z of the electrode. The contact surface 8z can extend arcuately and concentrically (i.e., can share the same centerpoint) with the interior surface 22 of the housing 4 in a reference plane orthogonal to the central axis X. The contact surface 8z can also have a curvilinear contour that is substantially conformal with the interior surface 22 in a direction along the central electrode axis 8x. The contact surfaces 8z can be smooth, as shown, although in other embodiments the contact surfaces 8z can be textured to enhance grip against the tissue 102, such as with protrusions, dimples, knurls, microneedles, and/or a roughened surface, by way of non-limiting examples. In additional embodiments, a coating or adhesive can be applied to the contact surfaces 8z to improve the grip and/or conductivity between the electrode 8 and the tissue 102. The electrodes 8 can also define a secondary portion 8f that extends from the contact portion 8e to the first end 8a and can be configured to connect with a respective lead of the circuitry 108 for transmitting the electroporation pulse(s) to the electrodes 8.

Referring now to FIG. 2D, the housing 4 can be include a housing body 26, which can be formed of a material that preferably has a measure of flexibility, such as a polymeric material, including polyetheretherketones (PEEK), polyphthalamides (PPA), polyethylenes, polycarbonates, polytherimides (PEI), polyvinyl chlorides (PVC), polytetrafluoroethylenes (PTFE), polyamides, polyimides, polysiloxanes (silicone), polyethylene terephthalates, polyurethanes, crosslinked or non-crosslinked rubbers (elastomers), polyesters, by way of non-limiting examples. It should be appreciated that other bio-compatible and/or medical-grade materials can be employed for the housing body 26. The housing body 26 can optionally be a monolithic structure that defines the housing 4, although the housing body 26 need not be a monolithic structure and can instead include two or more body components coupled together to define the housing 4. The housing body 26 extends from a proximal end 28 to the distal end 10 along the longitudinal direction L. The housing body 26 also defines a wall 30 that extends from the internal surface 22 to an external surface 32 of the housing 4. The wall 30 extends circumferentially around an entire perimeter of the vacuum chamber 6.

The housing body 26 defines the ports 12, 14, 16. As shown, each of the first, second, and third ports 12, 14, 16 can be adjacent the proximal end 28 of the housing body 26 and remote from the distal end 10. Stated differently, the ports 12, 14, 16 can be located closer to the proximal end 28 than to the distal end 10 of the housing 4. The first port 12, which can also be referred to as a “vacuum port,” extends from the vacuum chamber 6, through the housing body 26, and to a port coupling 34 for connection with a fitting member 36 that interconnects the vacuum port 12 with the vacuum source 106. The port coupling 34 can include a seat 38 and a tubular extension 40 that extends outwardly from the seat 38 and defines a receptacle, such that the seat 38 defines an inner end of the receptacle. The fitting member 36 can include a fitting member coupling 42 and a cannulated stem 44 extending therefrom. The fitting member coupling 42 can be a tubular extension that interconnects with the tubular extension 40 of the port coupling 34, such as by extending within the receptacle defined by the tubular extension 40 in mating fashion. A one-way valve member 46 can be positioned on the seat 38 (which can be referred to as a “valve seat”). The valve member 46 can extend from the valve seat 38 and within an interior space of the fitting member coupling 42, thereby being interposed in the fluid pathway between the vacuum port 12 and the cannulated stem 44 of the fitting member 36. The valve member 46 can be a duckbill valve, as shown, although in other embodiments the valve 46 can be ball valve or an umbrella valve, by way of non-limiting examples.

The second port 14 can extend opposite the first port 12 and can be configured to allow passage for the circuitry 108, such as wires, through the housing 4 and into contact with the electrodes 8 in the vacuum chamber 6. The second port 14 can also be configured to allow passage for one or more additional components, such as one or more tools and/or one or more sensors, through the housing 4 and into the vacuum chamber 6. While positioned inside the vacuum chamber 6, such tools and/or sensors can be positionally secured with respect to the tissue via vacuum pressure supplied to the chamber 6. The third port 16 can extend from the proximal end 24 of the chamber 6 and along the central axis X. The housing 4 can define a mounting formation 48 at an external end of the third port 16. The mounting formation 48 can be configured to mount a cap 50, such as a puncture stopper, over the third port 16. The mounting formation 48 and the puncture stopper 50 can have complimentary, mating geometries that provide an air-tight seal between the puncture stopper 50 and the third port 16. The puncture stopper 50 can be formed of a material that can be pierced by the hypodermic needle 18 allowing the needle 18 to inject the agent into the tissue 102 drawn into the vacuum chamber 6.

With continued reference to FIG. 2D, the electrode array 9 can be disposed on an insert 52, such as a sleeve, located in the chamber 6 and extending along the interior surface 22 of the housing 4. The sleeve 52, or at least an exterior surface 53 thereof, can have substantially the same profile geometry as the interior surface 22 of the housing 4. The sleeve 52 can optionally be constructed of a flexible material, such as rubber, silicone, and thermoplastic elastomers, by way of non-limiting examples. As shown in FIG. 2E, the exterior surface 53 of the sleeve 52 can adhere directly to the interior surface 22 of the housing 4 via a friction fit, although one or more adhesives can optionally be employed to attach the sleeve 52 to the interior surface 22. The sleeve 52 can extend from a first or proximal end 54 adjacent the proximal end 24 of the vacuum chamber 6 to a second or distal end 56 adjacent the distal end 10 of the housing 4. The distal end 56 of the sleeve 52 can extend within the lead-in portion 22a of the interior surface 22 of the housing 4. The first end 54 can define a proximal opening 52a of the sleeve 52, which can be concentric with the central axis X and a distal opening 52b of the sleeve 52. As shown more clearly in FIG. 2F, the first end 54 of the sleeve 52 can partially occlude the first port 12, and can also partially occlude the third port 16 while allowing passage for the circuitry 206 into the vacuum chamber 6. Thus, the sleeve 52 can be employed as a mechanism for controlling or at least affecting vacuum pressure within the chamber 6.

The vacuum chamber 6 defines a chamber volume V, which is defined between the proximal end 24 of the chamber 6 to the opening 10 along the longitudinal direction L, and can also be at least partially defined by the interior surface 22 of the housing body 26, such as along a direction substantially perpendicular to the longitudinal direction L. In the illustrated example, the direction perpendicular to the longitudinal direction L is a radial direction R that intersects the central axis X. The chamber volume V can also be at least partially defined by the sleeve 52, such as along the radial direction R. The chamber 6 can have a depth L2 measured from the proximal end 24 of the chamber 6 to a reference plane circumscribing the distal end 10 of the housing 4. The chamber depth L2 can be in a range from about 1.0 mm to about 50.0 mm, more particularly in a range of about 3 mm to about 20 mm, and more particularly in a range from about 5 mm to about 17 mm. The chamber 6 also has a base width, such as a base chamber diameter D1, which can be measured along the radial direction R adjacent the distal end 56 of the sleeve 52. The chamber diameter D1 can be in a range from about 1.0 mm to about 50.0 mm, particularly in a range of about 3.0 mm to about 20.0 mm, and more particularly in a range from about 6.0 mm to about 17.0 mm. In the present embodiment, the chamber diameter D1 can be measured between opposed portions of an interior surface 55 of the sleeve 52 near the distal end 56 thereof. In other embodiments, the sleeve 52 can be omitted and the electrodes 8 can be attached directly to the interior surface 22 of the housing 4, for example, by being embedded or at least partially embedded within the housing wall 30. In such embodiments, the vacuum chamber 6, and thus the chamber volume V, can be at least partially defined by the interior surface 22 of the housing 4 and the interior surfaces 8z of the electrodes 8. Accordingly, in such embodiments, the chamber diameter D1 can be measured between opposed portions of the interior surface 22 of the housing 4.

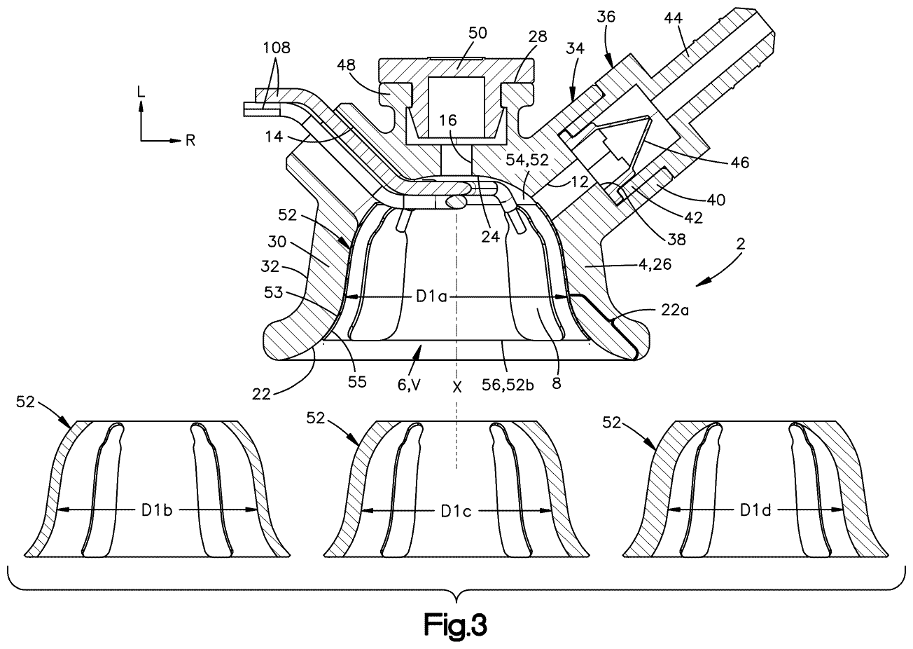

Referring now to FIG. 3, in some embodiments, the vacuum cup 2 can be provided in a kit that includes an outer housing 4 and a plurality of internal inserts 52 of different size that are interchangeably attachable to the interior surface 22 of the housing 4. For example, as shown, the internal inserts 52 can define different internal chamber diameters D1a, D1b, D1c, D1d. In this manner, the user can select the internal insert 52 having the preferred chamber diameter D1 for specific treatment factors, such as injectate volume, the anatomical treatment location, skin thickness, patient age, size, and body fat percentage, by way of non-limiting examples. In other embodiments, the interchangeable inserts 52 can have other differing characteristics, including electrode configuration, chamber geometry, and those more fully described in the '941 Reference. It should be appreciated that the chamber size (e.g., chamber diameter D1) can be correlated with respective injectate volumes. For example, the chamber diameter D1 can be chosen to correspond to a volume of tissue associated with holding a respective volume of injectate. In this manner, the insert 52 can be selected so that the chamber 6 can hold therein an associated volume of tissue that holds the injectate and can be expected to interact favorably to the applied vacuum pressure and optionally electroporation for causing transfection and/or immunogenicity.

Tests

The inventors conducted numerous tests to evaluate the efficacy of vacuum-assisted treatments, including vacuum-assisted treatments with electroporation and without electroporation. As discussed in the '941 Reference and as discussed below in supplementary fashion, the vacuum cups 2 can isolate a controlled volume of skin tissue and perform noninvasive EP to induce transfection and immunogenicity following intradermal injection of a DNA vaccine. Compared to other noninvasive EP methods, vacuum-assisted intradermal EP (ID-VEP) delivery is uniquely advantageous because suction can maintain intimate electrode-tissue contact for the duration of the procedure while isolating a large tissue volume, and the deformation of skin allows electric fields to propagate directly through the injection site and multiple tissue layers rather than following a shallow, lateral path along the skin surface. The impact of vacuum pressure, independent of EP, was also studied to isolate the impact of the regions of localized increased pressure within skin tissue on both transfection and immunogenicity. The tests herein used computational modeling to predict the impact of key ID-VEP design parameters on electric field distribution, and ID-VEP prototypes were built to evaluate in vivo gene expression and immunogenicity in guinea pigs. Immune studies were performed in guinea pigs using a DNA vaccine targeting MERS-CoV as a model system to demonstrate feasibility of this novel, needle-free electroporation system. Test data and results will now be described.

Device Design and Finite Volume Analysis (FVA)

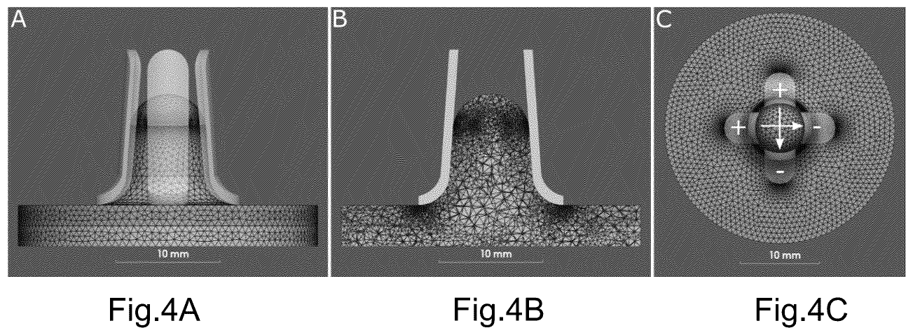

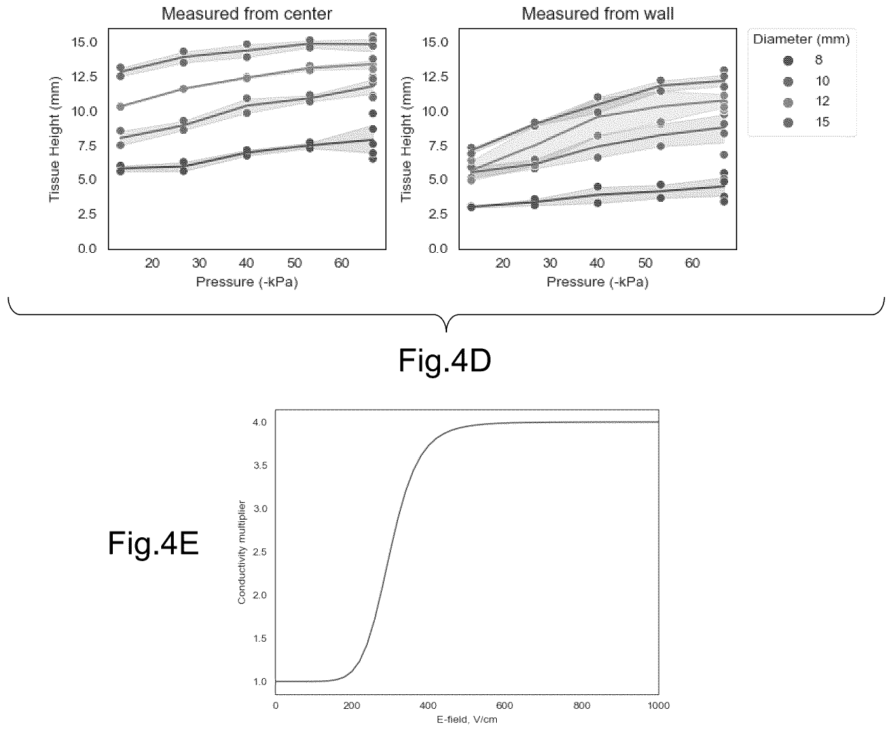

Referring now to FIGS. 4A-4C, an example virtual, meshed three-dimensional (3D) model of tissue is shown, with the tissue depicted as pulled within the chamber 6 of a vacuum cup 2, with electrodes 8 overlaid against the tissue. As shown in FIG. 4C, the pulse pattern (also referred to as “firing pattern”) of two unique pulses which were modeled between opposing electrode pairs. The depicted tissue model is based on a vacuum cup 2 having a chamber diameter D1 of 8 mm and configured for intradermal (ID) treatment via vacuum-assisted electroporation (VEP). Similar tissue models were also generated based on cup 2 designs having chamber diameters D1 of 10 mm, 12 mm, and 15 mm. These tissue models were generated using SolidWorks (SolidWorks Corp, Concord, MA, USA) to evaluate electric field distributions in tissue pulled into the various vacuum cups 2 designs at different operational conditions (e.g., vacuum pressure and electric pulse voltage). These 3D tissue models were used to create ID-VEP prototype vacuum cups 2, which were used for in vivo studies described herein. To create the virtual 3D model of tissue deformation within the vacuum chamber 6, each prototype cup 2 was placed against guinea pig skin, various vacuum pressures were applied, and the skin deformation was measured under each condition. As shown in FIG. 4D, the tissue heights were measured at both the center of the chamber 6 and at the wall 30 at various vacuum pressures. These measurements were used to create the 3D models of skin tissue for the ID-VEP cup 2 designs (see FIGS. 4A-4C), and these tissue models were used to perform finite volume analysis (FVA) to model electric field distribution within the tissue during electroporation (EP). FVA was performed in ANSYS Fluent (ANSYS Software, Canonsburg, PA, USA). The mesh for each 3D model was generated using the default ANSYS Fluent meshing algorithm.

To solve the model, skin was assigned an electrical conductivity of 0.17 S/m, and subcutaneous fat was assigned an electrical conductivity of 0.05 S/m. These properties were based on a publicly available database that aggregates experimental measurements of various tissue properties, as well as historical in-house readouts. Maxwell's equations were solved and a residual error of 10⁻⁷ was reached to achieve convergence. The potential difference between the active electrodes (200V for most models) was defined as a constant boundary condition. To model electric field-dependent increases in tissue conductivity, conductivities were progressively transformed based on electric field magnitude at each iteration using a sigmoid equation for a maximum increase of fourfold as electric field magnitude approached 600 V/cm, as shown in FIG. 4E. This technique has been previously described as a way to more accurately model the electric field-dependent changes that occur during electroporation in living tissue compared to models using constant conductivity values.

Animals

All in vivo studies were performed using female Hartley guinea pigs 12-16 weeks in age and weighing approximately 600 g. For the duration of all procedures, guinea pigs were maintained under anesthesia by inhaled isoflurane. Treatment sites were shaved immediately prior to each procedure. For terminal studies, euthanasia was performed by intracardiac injection of pentobarbital. All animal studies were performed under a protocol approved by an Institutional Animal Care and Use Committee.

Plasmid DNA Delivery and Electroporation

Intradermal plasmid delivery for all studies consisted of 100 μL ID injection of plasmid DNA onto the flanks of guinea pigs, using a 0.5 mL insulin syringe equipped with a 29-gauge needle. For ID-EP procedures, the CELLECTRA® ID Array—a minimally-invasive array containing three 3 mm needle electrodes—was immediately inserted into the injection site and EP was performed, delivering two sets of two pulses with an intensity of 0.2 A and a pulse duration of 52 msec. For ID-VEP, the vacuum chamber was placed on top of the injection site, the vacuum pump was powered on, and EP was performed once the pressure reached the set-point, delivering two sets of two pulses with an intensity of 200V and a pulse duration of 100 msec. After EP, the vacuum pump was turned off to release the tissue from the treatment chamber. The duration of the entire ID-VEP procedure, following intradermal DNA injection, was approximately 5-10 seconds, which is comparable to the duration of an ID-EP procedure. A GEMINI™ vacuum pump equipped with analog pressure control knob (VWR, Radnor, PA, USA) was used as a vacuum source for all studies. A custom pulse generator was used to deliver the EP pulses.

Gene Expression

Gene expression studies were conducted in guinea pigs to evaluate the breadth and magnitude of plasmid transfection following ID-VEP with different vacuum and electrical limits. In guinea pigs, ID-VEP delivery of plasmid encoding Green Fluorescent Protein (pGFP) at a concentration of 0.5 mg/mL was performed, using voltage limits of 50, 100, 100, or 200V at both low (−40 kPa) and high (−70 kPa) vacuum strengths. As a control, pGFP was delivered using injection alone (without vacuum), as well as via injection followed by −70 kPa vacuum (without EP). Skin was excised for analysis three days after plasmid delivery, and fluorescence was quantified using a FluorChem R imaging system (ProteinSimple, San Jose, CA, USA). Table 1 summarizes the experimental conditions and number of replicates. Injection sites were also photographed prior to tissue harvest to visually compare signs of superficial tissue damage.

Table 1. Gene expression studies. Guinea pigs received 100 μL intradermal injections of 0.5 mg/mL plasmid encoding GFP. Vacuum strength and voltage were varied. Injection alone, or injection with −70 kPa vacuum but no EP, were used as controls. Parameters of these gene expression studies are shown below in Table 1.

TABLE 1

| | | | Vacuum | |
|---|---|---|---|---|
| | | EP | strength | Voltage |
| Group | n | device | (kPa) | (V) |
| 1 | 4 | — | — | — |
| | | ID- | | |
| 2 | 8 | Vacuum | −70 | — |
| | | (no EP) | | |
| 3 | 8 | ID-VEP | −40 | 50 |
| 4 | 14 | ID-VEP | −70 | 50 |
| 5 | 8 | ID-VEP | −40 | 100 |
| 6 | 18 | ID-VEP | −70 | 100 |
| 7 | 8 | ID-VEP | −40 | 200 |
| 8 | 14 | ID-VEP | −70 | 200 |

Immunogenicity Studies

FOUR INDEPENDENT IMMUNOGENICITY STUDIES were performed in guinea pigs, each comparing ID-VEP to different delivery systems or different ID-VEP parameters. Vaccinations used 100 μL intradermal injection of 50 μg plasmid encoding MERS-CoV spike protein. ID-EP was performed using the CELLECTRA® 3P, a minimally invasive device that uses three needle electrodes, while ID-VEP was performed using VEP prototypes with parameters described in Table 2. Vaccinations were performed at week 0 and week 2 of each study, and an additional vaccination was performed at week 4 for two of the four studies.
a) STUDY 1 was designed to quantify the impact of both vacuum strength and electroporation (EP) voltage on vacuum-assisted electroporation (ID-VEP) immunogenicity.
b) STUDY 2 directly compared intradermal needle electroporation (ID-EP) to intradermal vacuum-assisted electroporation (ID-VEP),
c) STUDY 3 evaluated each component of ID-VEP (injection, vacuum, and EP) in an additive manner.
d) STUDY 4 compared different ID-VEP chamber diameters.
   These four studies shared a common study group using a 10-mm diameter ID-VEP cup 2 at 200 V, in order to verify the inter-study repeatability of the ID-VEP procedure. Serum was collected every two weeks to measure humoral response kinetics via ELISA, and whole blood was collected at week 4 for ELISPOT analysis. All treatments were performed in guinea pigs, using 100 μL intradermal injection of plasmid encoding MERS-CoV spike protein followed immediately by electroporation. Parameters for these four studies are shown below in Table 2.

TABLE 2

| | | | | VEP | Vacuum | Voltage | Vaccine schedule |
|---|---|---|---|---|---|---|---|
| Study | Group | n | Delivery | diameter (mm) | (kPa) | (V) | (weeks) |
| 1 | 1 | 5 | ID-VEP | 10 | −40 | 100 | 0, 2 |
| | 2 | 5 | ID-VEP | 10 | −70 | 100 | |
| | 3 | 5 | D-VEP | 10 | −70 | 200 | |
| 2 | 1 | 5 | ID-VEP | 10 | −70 | 200 | 0, 2, 4 |
| | 2 | 5 | ID-EP | — | — | 200 | |
| 3 | 1 | 5 | ID-VEP | 10 | −70 | 200 | 0, 2 |
| | 2 | 5 | ID-Vac (no EP) | 10 | −70 | — | |
| | 3 | 5 | ID (no EP) | 1 | — | — | |
| 4 | 1 | 5 | ID-VEP | 8 | −70 | 200 | 0, 2, 4 |
| | 2 | 5 | ID-VEP | 10 | −70 | 200 | |
| | 3 | 5 | ID-VEP | 12 | −70 | 200 | |
| | 4 | 5 | ID-VEP | 15 | −70 | 200 | |

ELISA

ELISA was performed as described previously in P. D. Fisher et al., “Adipose tissue: a new target for electroporation-enhanced DNA vaccines,” Gene Therapy, vol. 24, no. 12, Art. no. 12, December 2017, doi: 10.1038/gt.2017.96. Briefly, 96-well plates (Thermo Fisher) were coated overnight with MERS spike protein antigen (Sino Biological, 40069-V08B) in PBS. Plates were then blocked with 3% bovine serum albumin (BSA) for 2 hours at room temperature (RT) and washed. Serum in PBS-t containing 1% BSA (Millipore Sigma) was added for 2 hours at RT and then washed, and then HRP-conjugated goat anti-guinea pig IgG (Millipore Sigma, A7289) was incubated in wells for 1 hour at RT. Tetramethylbenzidine substrate solution (VWR) was used to develop color for 6 minutes, then methylbenzidine stop reagent solution (VWR) was added to halt the reaction. A Synergy HTX plate reader (BioTek Instruments) was used to measure absorbance at 450 nm.

Endpoint binding titers were defined as the intersection point between a 5-parameter logistic curve for each biological replicate at each time point and a single reference curve fitted to the upper limit of the 99% prediction interval calculated at each dilution for all week 0 samples.

ELISPOT

ELISPOT was performed as described previously in Fisher et al., “Adipose tissue: a new target for electroporation-enhanced DNA vaccines” (supra); K. Schultheis et al., “Optimized Interferon-gamma ELISpot Assay to Measure T Cell Responses in the Guinea Pig Model After Vaccination,” Journal of Visualized Experiments, January 2018, doi: 10.3791/58595; and K. Schultheis et al., “Characterization of guinea pig T cell responses elicited after EP-assisted delivery of DNA vaccines to the skin,” Vaccine, vol. 35, no. 1, pp. 61-70, January 2017, doi: 10.1016/j.vaccine.2016.11.052. At 2, 4, and/or 6 weeks following initial vaccination, 3 mL of peripheral blood was collected. Peripheral blood mononuclear cells (PBMCs) were isolated and plated at a density of 1×105 cells per well on X-coated 96-well Millipore IP plates. Plated PBMCs were stimulated with 5 MERS peptide pools (GenScript) and IFN-γ production was measured using biotinylated mouse monoclonal anti-guinea pig IFN-γ antibody and BCIP/NBT detection reagent substrate. A CTL-Immunospot S6 ELISPOT plate reader and its included software was used to count spot-forming units (SFU). Spot counts were normalized to baseline by subtracting SFU of wells that did not receive any peptide.

Statistical Analysis

Prior to analysis, gene expression data were converted from raw fluorescence values with arbitrary units into ratios relative to a reference group. All immunogenicity data—binding titers for antibody responses and SFU for cellular responses—were log-transformed prior to analysis. For direct comparison of two study groups, a t-test with Welch correction was performed. Otherwise, simple linear regression or multiple linear regression were performed at each time point of each study. Since this work is exploratory in nature and the objective of these studies was to generate new hypotheses for future work, multiplicity corrections which account for Type I error but may increase Type II error were not performed.

Results

By way of summary, the results described below demonstrate, among other things, that DNA vaccines delivered via ID-VEP are highly immunogenic—comparable to needle-based ID-EP—and the delivery procedure itself is noninvasive, repeatable, and requires only a few seconds to perform. Design parameters of the vacuum cup 2, such as chamber size, pulse intensity, and vacuum strength, were shown to influence DNA vaccine immunogenicity and gene expression, and the use of FVA modeling supports the hypothesis that the breadth and magnitude of the electric field can predict and explain performance of ID-VEP systems. Negative pressure was shown to independently enhance transfection and synergized with EP delivery by immobilizing a fixed volume of skin securely against the electrodes lining the EP chamber. This technique provides a reliable, repeatable platform to perform noninvasive EP, and the impact of negative pressure on skin tissue and its interaction with electroporation merits continued research. More specific results are described below.

FVA and Prototype Development

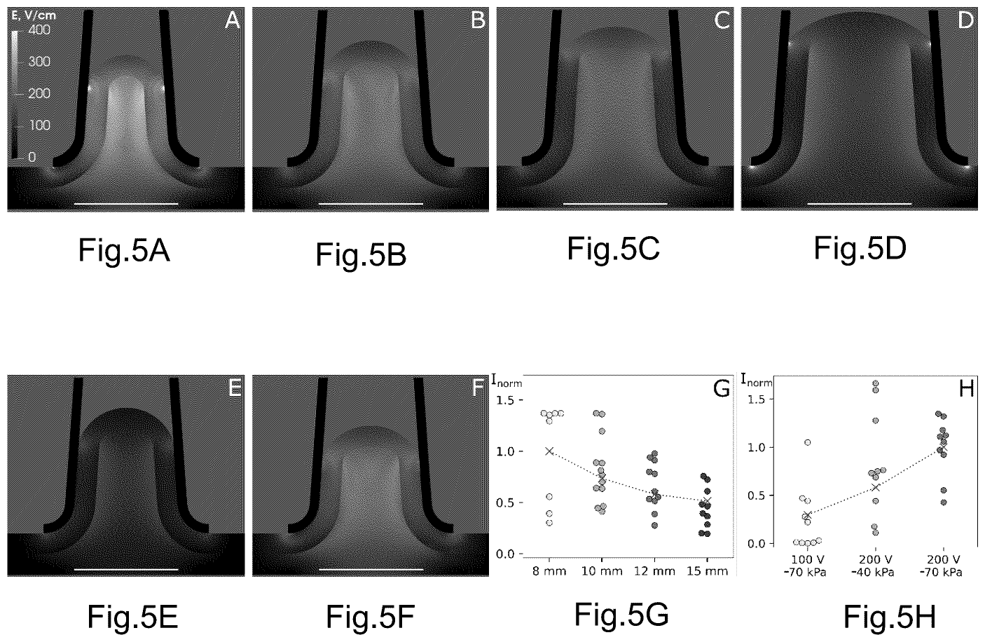

With reference to FIGS. 5A-5F, FVA was performed to investigate the impact of the following parameters on electric field distribution: pulse intensity, ID-VEP chamber diameter, and vacuum strength. Paraview software was used to generate the visualized electric field distributions of each solved model shown in FIGS. 5A-5F. The boundary between the skin layer 104 and adipose layer 103 is visible; the white scale bar represents 10 mm in length; and the FVA predictions were normalized to the mean electric current of a reference group in each study. In FIGS. 5A-5F, ID-VEP prototype cups 2 with chamber diameters D1 measuring 8 mm (FIG. 5A), 10 mm (FIG. 5B), 12 mm (FIG. 5C), and 15 mm (FIG. 5D) were modeled based on an applied vacuum pressure of −70 kPa and an electrical pulse intensity of 200 V between active electrodes 8. In FIGS. 5E-5F, the electric field distributions were calculated for vacuum cups 2 having a chamber diameter of D1=10 mm and vacuum-electrode parameters of −70 kPa and 100 V (FIG. 5E) and −40 kPa and 200 V (FIG. 5F).

In each of FIGS. 5A-5F, the ID-VEP cup 2 designs generate relatively uniform electric fields to all tissue contained inside the vacuum chamber 6, with slightly higher field strengths observed within subcutaneous adipose tissue 103 as well as small “hot spots” where skin tissue 104 loses contact with the electrodes 8. There was a clear inverse association between electric field intensity and chamber diameter D1 for simulations run at 200V and −70 kPa vacuum strength (FIGS. 5A-5D). For the cup 2 having a chamber diameter D1 of 10 mm (FIGS. 5B and 5E-5F), decreasing voltage to 100V appeared to dramatically decrease the electric field strength, while decreasing the vacuum strength to −40 kPa did not visually alter electric field strength but did result in a smaller volume of tissue contained within the vacuum chamber 6.

Referring now to FIGS. 5G-51I, the ID-VEP cup 2 designs and parameter sets shown in FIGS. 5A-5F were prototyped and tested in vivo following DNA vaccination in guinea pigs. The in vivo electrical data collected during the ID-VEP cup 2 DNA vaccinations was compared to the predicted, modeled electrical output of each ID-VEP cup 2 configuration to evaluate the accuracy of FVA simulations shown in FIGS. 5A-5F. In FIG. 5G, the ID-VEP prototype cups 2 with chamber diameters D1 of 8 mm, 10 mm, 12 mm, and 15 mm were tested to compare the measured in vivo electrical parameters in tissue for comparison to the modeled electrical parameters. In FIG. 511, the ID-VEP prototype cup 2 with a chamber diameter D1 of 10 mm was tested for three different combinations of pulse intensity and vacuum strength: (1) 200 V and −70 kPa; (2) 100V and −70 kPa; and (3) 200V and −40 kPa for comparison to the modeled results.

These results demonstrate that, after normalizing the FVA predictions to the mean in vivo current of a reference group for each study, the FVA was capable of accurately predicting the impact of ID-VEP chamber diameter D1, pulse voltage, and vacuum strength on delivered current. As shown in FIG. 5G, larger ID-VEP chamber diameters D1 resulted in progressively lower measured and predicted electric current for a pulse voltage of 200V, spanning a 50% reduction in current as ID-VEP diameter D1 was increased from 8 mm to 15 mm. Additionally, as shown in FIG. 511, the 10 mm ID-VEP prototype cup 2 exhibited an approximately 50% reduction in current when vacuum strength was reduced from −70 kPa to −40 kPa for a pulse voltage of 200V, while current was reduced by 75% when pulse voltage was reduced to 100V and vacuum strength was maintained at −70 kPa. Notably, the 100 V condition did not consistently deliver pulses (several treatments had a mean current close to 0 Amps).

These results also demonstrate that FVA can be used to prospectively screen the performance of ID-VEP prototype cups 2 and devices and can serve to both inform future design decisions and explain experimental readouts. Reducing pulse voltage from 200V to 100V resulted in electric field magnitudes which were generally less than 100 V/cm, which is below the threshold typically associated with successful transfection, although modeling noninvasive electroporation of skin has been historically difficult due to the less-understood contributions of the stratum corneum. Smaller ID-VEP diameters showed dramatically stronger field strengths for a given voltage, suggesting that the impact of reduced electrode surface area is outweighed by the reduction in inter-electrode spacing. Despite the complex geometry and simplifying assumptions used to build these models, it is promising that the electrical impact of various delivery parameters predicted by FVA closely matches the electrical trends observed in vivo, and these results suggest that FVA may have predictive value when evaluating ID-VEP designs. Furthermore, immune responses (discussed below) also correlated with the observations from simulated electric fields: stronger responses were associated with more intense electric fields.

The FVA performed here assumed that each tissue layer is homogeneous, with electrical conductivity that increases in a sigmoid fashion with electric field strength. Additionally, the stratum corneum was excluded from the model because very thin features can introduce artifacts in finite volume models, and skin was assumed to be a single layer rather than a multilayered composite material. Lastly, thermal effects were not considered in this model. Because these models consistently overestimated total electric current compared to in vivo, it is possible that further optimization of the conductivity transformation and inclusion of the stratum corneum may yield more accurate results. Once normalized to the baseline in vivo readouts, the models were quite accurate and provide evidence that electric field distributions predicted by these simulations can model ID-VEP device performance.

Gene Expression

Studies evaluating in vivo GFP expression of ID-VEP in guinea pigs indicated that stronger vacuum pressure enhances gene expression regardless of EP voltage. For example, with reference to FIG. 36B of the '941 Reference, a combination of 100 V ID-VEP voltage and −70 kPa vacuum strength produced the strongest overall GFP expression on the skin surface in that test. With continued reference to FIG. 36B of the '941 Reference, across injection sites receiving electroporation, increasing vacuum pressure from −40 kPa to −70 kPa doubled GFP expression. Additionally, increasing EP voltage from 50 V to 100 V resulted in a 1.4-fold increase in expression, while further increasing from 100 V to 200 V reduced visible expression by nearly 70%. In the absence of electroporation, briefly applying −70 kPa vacuum pressure to the injection site resulted in more than threefold higher GFP expression on the skin surface than intradermal injection alone (Welch t-test: Fluorescence ratio (95% CI)=3.39 (1.16, 5.62), p=0.038).

With reference to FIG. 36A of the '941 Reference, in addition to quantification of GFP fluorescence, expression patterns at treatment sites receiving high vacuum strength were photographed and visually compared. ID injection of pGFP caused mild erythema even in the absence of vacuum or EP, and resulting GFP expression was concentrated into a small, centralized region. The addition of −70 kPa vacuum strength alone created a broader, circular region of both erythema and transfection, and the further addition of ID-VEP voltages up to 100V increased the intensity, but not the breadth, of fluorescent signal. At 200V, the regions with the darkest erythema corresponded with a complete absence of fluorescence signal.

Parameters involved with the tests shown in FIGS. 36A-36B of the '941 Reference are further identified in Table 3 below. In Table 3, multiple linear regression of superficial GFP expression following ID delivery with combinations of ID-VEP vacuum strengths and voltages, using vacuum pressure and voltage as predictors of fluorescence. Backward difference contrast coding was used for the Voltage variable to provide progressive comparisons between increasing voltage groups.

TABLE 3

| Dependent | Independent | | | |
|---|---|---|---|---|
| variable | variable | Coefficient | 95% CI | p-value |
| Fluorescence | Vacuum strength | 2.00 | (1.47, 2.55) | <0.001 |
| ratio | Voltage | 1.41 | (1.05, 1.76) | 0.026 |
| | (50 vs 100 V) | | | |
| | Voltage | −0.31 | (−0.06, −0.55) | <0.001 |
| | (100 vs 200 V) | | | |

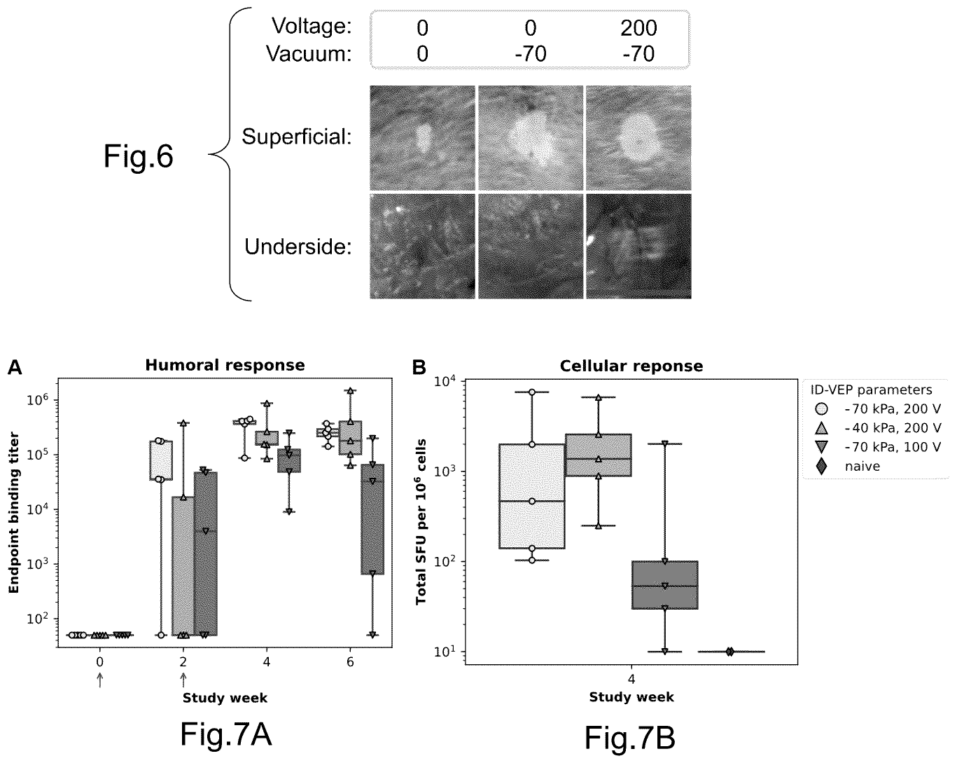

Referring now to FIG. 6, gene expression was also detectable in underlying skin layers following ID-VEP in some samples, but was not detectable in the absence of EP in any samples, even when vacuum was applied to the injection site. In FIG. 6, imaging shows superficial and underside of skin samples harvested three days following plasmid delivery via intradermal injection alone, intradermal injection followed by vacuum at −70 kPa, or intradermal injection followed by ID-VEP with vacuum strength of −70 kPa and EP voltage of 200 V. Note the underside expression at 200 V and −70 kPA (bottom right) shows a striated pattern typical of panniculus carnosus muscle, which resides below the subcutaneous fat layer. The scale bar represents 10 mm in length.

The gene expression analysis herein revealed potential synergy between vacuum and EP effects on local protein production. Even in the absence of EP, application of vacuum pressure transfected a large region of superficial skin cells. This change in expression breadth suggests that the skin deformation caused is physically disrupting cell membranes and transfecting cells, and possibly redistributing plasmid laterally through the skin to physically contact more cells. The addition of EP showed a voltage-dependent increase in GFP fluorescence intensity, but not breadth, suggesting that the two techniques may be complementary: vacuum can increase the area of transfection, while EP delivers more plasmid copies over the same area. While superficial expression is primarily due to epidermal skin cells, imaging the underside of skin sections highlights expression in the deeper dermal layers, the subcutaneous adipose tissue, and the panniculus carnosus which is present in rodent skin. Although expression in the underlying layers was not detectable in all samples, it is notable that expression in deeper tissue layers was only detectable at treatment sites that had received electroporation. This finding suggests that noninvasive ID-VEP is capable of transfecting superficial as well as deeper layers within skin tissue—similar to ID-EP devices that use needle electrodes—whereas intradermal injection alone, with or without vacuum, is limited mainly to the superficial epidermis. Future ID-VEP studies should investigate these differences in protein expression patterns at the histological level in order to more fully understand the spatial distribution of transfection and the cell populations involved.

Interestingly, when voltage was increased from 100V to 200V, superficial GFP expression in skin sharply declined. This decrease can be attributed to more prominent erythema associated with skin irritation due to higher pulse energy, which effectively quenches visible GFP signal. Therefore, superficial GFP expression readouts are probably unreliable when higher pulse energies are employed, since redness of the tissue may be masking positive signal. It was encouraging to note that none of the ID-VEP treatment sites exhibited any visible signs of irritation beyond transient erythema at the injection site, even at high voltages. These promising initial results suggest that ID-VEP is well-tolerated and causes minimal tissue damage, although future studies should be conducted to more thoroughly characterize the tissue-level and cell-level responses to the ID-VEP procedure.

Immunogenicity

Study 1

Referring now to FIGS. 7A-7B, the immunological impact of ID-VEP vacuum strength and voltage was quantified using humoral and cellular readouts in guinea pigs vaccinated against MERS-CoV using ID-VEP delivery. Two 50 μg vaccinations were performed at study weeks 0 and 2. In FIG. 7A, humoral response kinetics of different ID-VEP parameter combinations are shown. Arrows indicate treatments. In FIG. 7B, cellular response measured at week 4 following initial vaccination. IFN-γ spot-forming units (SFU) are presented as the sum of five peptide pools' individual responses.

All animals (15/15) in this study seroconverted after the second vaccination, while after a single vaccination the seroconversion rates were 4/5 for −70 kPa/200V, 2/5 for −40 kPa/200V, and 3/5 for −70 kPa/100V (FIG. 7A). At week 4, when peak immunogenicity was observed, higher EP voltage was significantly associated with stronger binding titers, but higher vacuum strength was not. At week 6—four weeks after the second vaccination—titers had begun to decrease substantially in animals receiving 100V delivery, while they remained steady or only slightly decreased in animals receiving 200V at either vacuum strength. The impact of voltage and vacuum strength on cellular responses was measured via ELISPOT performed at week 4 of the study (FIG. 7B), which indicated that 200V delivery increased mean cellular responses by approximately tenfold compared to 100V, although this effect was not significant. Similar to humoral responses, there was minimal if any impact from vacuum strength on cellular response.

Study 2

With reference to FIGS. 37A-37B of the '941 Reference, the results of a head-to-head immunogenicity study are shown between ID-EP using intradermal needle electrodes and needle-free ID-VEP. Guinea pigs received pMERS DNA vaccine delivered using ID-EP or ID-VEP. A total of three 50 μg vaccinations were performed, one every two weeks beginning at week 0. In FIG. 37A of the '941 Reference, humoral response kinetics of ID-EP and ID-VEP delivery methods are shown, with arrows indicating treatments (although this Figure shows midpoint binding titers, endpoint binding titers were also measured and charted). In FIG. 37B of the '941 Reference, cellular responses are shown, measured at week 2 and week 4 following initial vaccination. IFN-γ spot-forming units (SFU) are presented as the sum of five peptide pools' individual responses.

As demonstrated in this study, ID-VEP appeared to generate a more rapid humoral response than ID-EP (FIG. 37A of the '941 Reference). By 2 weeks following the first vaccination, all animals in both groups had seroconverted and ID-VEP was associated with significantly higher titers than ID-EP. At subsequent time points following additional vaccinations, there was no significant difference between ID-VEP and ID-EP. Cellular responses, measured at week 2 and week 4 of the study (FIG. 37B of the '941 Reference), increased substantially after the second vaccination for each delivery method, and spot counts were comparable for ID-EP and ID-VEP at each time point.

Study 3

With reference to FIGS. 39A-39B of the '941 Reference, a separate study was conducted to evaluate the isolate the impact of vacuum pressure alone (ID-Vacuum) on immunogenicity. Guinea pigs received a pMERS vaccine delivered using three different modes: (1) using an ID-VEP cup 2, (2) ID delivery followed by vacuum fixation using a cup 2 (but without EP); and (3) ID delivery without vacuum or EP. Two 50 ug vaccinations were performed at study weeks 0 and 2. In FIG. 39A of the '941 Reference, humoral responses are shown, with arrows indicating treatments (although this Figure shows midpoint binding titers, endpoint binding titers were also measured and charted). In FIG. 39B of the '941 Reference, cellular responses are shown, measured at week 4. IFN-γ spot-forming units (SFU) are presented as the sum of five peptide pools' individual responses.

As demonstrated in this study, ID-VEP was associated with faster and stronger humoral immune responses compared to ID-Vacuum or ID delivery alone. After one vaccination, all ID-VEP animals (5/5) had seroconverted, compared 1/5 for ID-Vacuum and 0/5 for ID delivery without vacuum or EP. By week 4—two weeks after a second vaccination—each of the ID-Vacuum and ID groups showed seroconversion in 3/5 animals. The presence of electroporation was significantly associated with higher titers at week 2, although this effect was not significant at week 4 due to the highly variable, partial responses in the ID and ID-Vacuum groups. In contrast, the use of vacuum alone did not generate any measurable difference in endpoint titers compared to ID delivery alone at either time point, although it is notable that the two overall strongest responses at week 4 both received ID-Vacuum delivery. Cellular responses were measured at week 4 (FIG. 39B of the '941 Reference), and the use of electroporation was significantly predictive of higher spot counts while the use of vacuum alone was not.

Study 4

With reference to FIGS. 35A-35B of the '941 Reference, a separate study was conducted to evaluate the impact of ID-VEP chamber diameter on immunogenicity. Guinea pigs received a pMERS DNA vaccine delivered using ID-VEP cups 2 with chamber diameters D1 of 8 mm, 10 mm, 12 mm, and 15 mm. A total of three vaccinations were performed, beginning at week 0. In FIG. 35A of the '941 Reference, humoral responses are shown, measured every two weeks for 6 weeks, with arrows indicating ID-VEP treatments (although this Figure shows midpoint binding titers, endpoint binding titers were also measured and charted). In FIG. 35B of the '941 Reference, cellular responses are shown, measured at week 4 and week 6 following initial vaccination. Spot-forming units (SFU) are presented as the sum of five peptide pools' individual responses

As demonstrated in this study, ID-VEP chamber diameter D1 was inversely associated with humoral immune responses, with the smallest (8 mm) diameter generally providing the strongest and most rapid responses. The relationship between ID-VEP diameter and log-transformed binding titers was significant at each time point, and each millimeter of decreasing ID-VEP diameter was associated with a geometric mean endpoint titer increase of approximately 0.2 at week 2, 0.1 at week 4, and 0.09 at week 6. Two weeks after the initial vaccination, the response appeared to be plateauing for the 8 mm and 10 mm diameters. Cellular responses, collected by IFN-ELISpot two weeks after the second and third vaccinations, were generally the strongest for ID-VEP chamber diameters ranging from 8 mm to 12 mm, and increasing the diameter to 15 mm appeared to be associated with lower, more variable spot counts, although the impact of diameter on spot counts was not significantly predictive (see FIG. 35B of the '941 Reference).

A summary of test parameters and result parameters of the foregoing Studies 1-4 is provided below in Table 4. Linear regression models were used for statistical analysis of each immunogenicity study. At each week of each study, a linear regression model was fitted to ELISA and ELISpot readouts, and in cases where multiple independent variables are listed, multiple linear regression was performed. Significant results (p<0.05) are bolded.

TABLE 4

| | Dependent | | | | | |
|---|---|---|---|---|---|---|
| Study | variable | Week | Independent variable | Coefficient | 95% Cl | p-value |
| 1 | log₁₀(titer) | 2 | Voltage (100 vs 200 V) | 0.97 | (−1.23, 3.18) | 0.356 |
| | | | Vacuum strength (−40 vs −70 kPa) | 1.28 | (−0.93, 3.48) | 0.231 |
| | | 4 | Voltage (100 vs 200 V) | 0.64 | (0.07, 1.22) | 0.031 |
| | | | Vacuum strength (−40 vs −70 kPa) | 0.14 | (−0.43, 0.72) | 0.603 |
| | | 6 | Voltage (100 vs 200 V) | 1.55 | (0.29, 2.81) | 0.020 |
| | | | Vacuum strength (−40 vs −70 kPa) | 0.04 | (−1.22, 1.30) | 0.951 |
| | log₁₀(SFU) | 4 | Voltage (100 vs 200 V) | 0.90 | (−0.12, 1.92) | 0.079 |
| | | | Vacuum strength (−40 vs −70 kPa) | −0.34 | (−1.36, 0.68) | 0.480 |
| 2 | log₁₀(titer) | 2 | Device (ID-EP vs ID-VEP) | 0.41 | (0.07, 0.75) | 0.023 |
| | | 4 | Device (ID-EP vs ID-VEP) | 0.14 | (−0.19, 0.48) | 0.345 |
| | | 6 | Device (ID-EP vs ID-VEP) | 0.02 | (−0.40, 0.43) | 0.927 |
| | log₁₀(SFU) | 2 | Device (ID-EP vs ID-VEP) | 0.23 | (−1.01, 1.47) | 0.676 |
| | | 4 | Device (ID-EP vs ID-VEP) | −0.07 | (−0.39, 0.25) | 0.608 |
| 3 | log₁₀(titer) | 2 | EP (0 vs 200 V) | 1.88 | (0.51, 2.67) | 0.011 |
| | | | Vacuum (0 vs −70 kPa) | 0.74 | (−0.63, 2.10) | 0.371 |
| | | 4 | EP (0 vs 200 V) | 1.65 | (−0.86, 4.16) | 0.178 |
| | | | Vacuum (0 vs −70 kPa) | 0.40 | (−2.68, 3.49) | 0.798 |
| | log₁₀(SFU) | 4 | EP (0 vs 200 V) | 1.23 | (0.17, 2.30) | 0.027 |
| | | | Vacuum (0 vs −70 kPa) | −0.01 | (−1.08, 1.05) | 0.980 |
| 4 | log₁₀(titer) | 2 | ID-VEP diameter (mm) | −0.19 | (−0.34, −0.04) | 0.014 |
| | | 4 | ID-VEP diameter (mm) | −0.10 | (−0.17, −0.02) | 0.015 |
| | | 6 | ID-VEP diameter (mm) | −0.086 | (−0.12, −0.05) | <0.001 |
| | log₁₀(SFU) | 4 | ID-VEP diameter (mm) | −0.069 | (−0.16, 0.02) | 0.12 |
| | | 6 | ID-VEP diameter (mm) | −0.064 | (−0.15, 0.03) | 0.154 |

In these immunogenicity studies, ID-VEP was shown to be highly immunogenic, and the magnitude of gene expression and immune responses was directly influenced by the device geometry, the EP pulse parameters, and the vacuum pressure. ID-VEP chamber diameters D1 of from 8-10 mm are preferable based on these results, and only a few additional millimeters of size dramatically reduced immune responses.

Recently, a method of suction-mediated transfection to skin tissue was shown to increase localized transfection and immunogenicity of DNA vaccines in rats, as published in E. O. Lallow et al., “Novel suction-based in vivo cutaneous DNA transfection platform,” Science Advances, vol. 7, no. 45, p. eabj0611, doi: 10.1126/sciadv.abj0611. Interestingly, while the results disclosed above agree with the Lallow et al. findings that localized transfection is enhanced through negative pressure alone, the tests described in the present disclosure could not reproduce the finding that negative pressure alone also increases immunogenicity of DNA vaccines. Rather, the results herein show in guinea pigs that there was no meaningful enhancement in immunogenicity through suction alone, and only the addition of EP led to a dramatic improvement in humoral and cellular immune responses. This may be due to the choice of species for these two different studies; rats have substantially thinner stratum corneum, viable epidermis, and dermis thickness than human skin, whereas guinea pigs have skin more structurally similar to humans, with similarly thick epidermal and dermal layers. These divergent findings highlight the importance of multiple animal models to understand the complex phenomena involved in vacuum-mediated DNA delivery, and also suggest that the addition of electroporation may be necessary when delivering DNA vaccines to thicker, more human-like skin.

Human skin thickness (epidermis+dermis) ranges from 0.5 mm to 2 mm, which explains the design of existing ID-EP systems that target the first few millimeters of skin tissue. However, several studies have shown that subdermal cells can be transfected during ID-EP, and prior work from this group showed that subcutaneous fat contributes strongly to DNA vaccine immunogenicity even in the absence of epidermal or dermal transfection. For these reasons, ID-VEP is a rational next step in EP device design, because it can easily immobilize a controlled volume of skin and subcutaneous fat and perform electroporation across that entire volume noninvasively.

In the four immunogenicity studies herein, ID-EP parameters of 200 V pulse intensity and −70 kPa vacuum strength generated the strongest humoral and cellular responses compared to lower vacuum or voltage settings, which agrees with FVA predictions and contrasts with the gene expression findings as explained above. These results raise the possibility that even higher voltages than 200 V may continue to enhance immunogenicity, though it should be considered that increased pulse intensities are also associated with discomfort, thermal damage, and irreversible electroporation. Because the ID-VEP techniques described herein are intended to be highly-tolerable procedures causing no lasting tissue damage, 200 V was selected as a rational upper limit for these studies, though higher voltages merit further investigation since ID-VEP even at 200 V caused only transient irritation. In addition to pulse intensity alone, future work should also consider other combinations of pulse widths and inter-pulse delays in order to provide a more complete picture of how each electrical parameter impacts immunogenicity and superficial tissue damage.

In a head-to-head comparison between ID-VEP and needle-based ID-EP, ID-VEP generated equivalent cellular responses and superior humoral response kinetics and magnitude through 6 weeks of observation. This improvement may be attributable to the broader, more homogeneous electric field that is generated in ID-VEP compared to needle ID-EP. The reason for such effective electric field coverage is the vacuum component of ID-VEP, which positions skin tissue such that electric fields penetrate through all skin layers along two perpendicular axes. These results suggest that ID-VEP overcomes some of the challenges associated with noninvasive EP devices—namely, shallow and inconsistent electric field generation—which have generally led to lower immunogenicity compared to needle-based ID-EP.

Although ID-Vacuum (without EP) delivery generated substantially higher GFP expression than ID delivery (without vacuum or EP), both methods resulted in comparably poor immunogenicity, with virtually no measurable humoral response after 1 vaccine and partial seroconversion after a second vaccination. In these studies, only the combination of vacuum pressure and electroporation was capable of inducing consistent, high-magnitude cellular and humoral responses. This result suggests that transfection alone may not sufficiently predict immune responses to DNA vaccines. Based on these findings, it is proposed that the combination of transfection breadth (due to vacuum pressure) and transfection efficiency (due to EP) act synergistically to increase overall gene expression, while EP is the dominant factor driving immunogenicity. Previous studies have shown that EP can be synergistically or additively combined with other delivery methods such as sonoporation, particle delivery, and jet injection. In addition to its well-characterized enhancement of gene expression, which was corroborated in this study, EP has also been theorized to act as a physical adjuvant since electrical energy produces localized tissue damage which can prompt the localized recruitment of innate and adaptive immune cell types, including macrophages and T-cells. This adjuvant effect may further explain the superiority of ID-VEP compared to ID-Vacuum delivery despite their similar gene expression profiles, and it may also explain why smaller ID-VEP diameters (which had stronger electric fields and more electrical “hot spots”) were associated with stronger immune responses.

The ID-VEP cup 2 prototypes described herein were designed and built with the objective of encompassing sufficient skin and subcutaneous tissue to transfect an entire 100 μL injection site, but not gathering so much tissue that excess tissue is electroporated needlessly. Therefore, most studies were performed using a 10 mm diameter ID-VEP cup 2 having a chamber diameter D1 of 10 mm, which is similar to the diameter of a standard 100 μL intradermal fluid bleb. The smallest ID-VEP chamber diameter D1 tested was 8 mm, and smaller sizes were not tested because the entire injection volume would be unable to fit inside the EP chamber. It should be appreciated, however, that larger or smaller ID-VEP devices may be appropriate for different injection volumes or desired depths of transfection.

The work described here used a MERS-CoV DNA vaccine as a model plasmid to develop the ID-VEP system; this research is now of particular relevance due to the ongoing COVID-19 pandemic caused by another coronavirus, SARS-CoV-2. It is encouraging, therefore, that ID-VEP appears to induce rapid and potent humoral responses, which have shown to correlate with protection from infection, hospitalization, and death, as well as strong cellular responses, which we hypothesize will be critical to provide long-lasting and adaptive protection against current and future mutations. A well-rounded immune response, generated with a noninvasive, easy-to-use, and well-tolerated ID-VEP device, is an attractive product profile in these pandemic situations where widespread vaccination is required.

Although the disclosure has been described in detail, it should be understood that various changes, substitutions, and alterations can be made herein without departing from the spirit and scope of the invention as defined by the appended claims. For example, features of the various embodiments described herein can be incorporated into one or more and up to all of the other embodiments described herein. Moreover, the scope of the present disclosure is not intended to be limited to the particular embodiments described in the specification. As one of ordinary skill in the art will readily appreciate from that processes, machines, manufacture, composition of matter, means, methods, or steps, presently existing or later to be developed that perform substantially the same function or achieve substantially the same result as the corresponding embodiments described herein may be utilized according to the present disclosure.

What is claimed:

1. A device for vacuum-assisted in vivo electroporation of tissue, comprising:
a housing defining a receptacle and at least one opening into the receptacle;
at least one port extending through the housing, wherein the at least one port is remote from the at least one opening and is connectable to a vacuum source, such that the at least one port is configured to communicate vacuum pressure from the vacuum source to the receptacle;
an insert receivable within the receptacle, the insert defining an internal chamber that is in communication with the vacuum source when the insert is received in the receptacle, the insert including:
a plurality of electrodes positioned within the chamber, wherein the plurality of electrodes are configured to deliver one or more electroporation pulses to a targeted portion of tissue extending through the at least one opening and at least momentarily held in the chamber responsive to the vacuum pressure.

2. A kit for vacuum-assisted in vivo electroporation of tissue, comprising:
a housing defining a receptacle and at least one opening into the receptacle;
at least one port extending through the housing, wherein the at least one port is remote from the at least one opening and is connectable to a vacuum source, such that the at least one port is configured to communicate vacuum pressure from the vacuum source to the receptacle;
a plurality of inserts that are interchangeably receivable within the receptacle, each insert defining an internal chamber that is in communication with the vacuum source when the insert is received in the receptacle, wherein the plurality of inserts define different respective chamber diameters, each insert including:
at least one electrode positioned within the chamber, wherein the at least one electrode is configured to deliver one or more electroporation pulses to a targeted portion of tissue extending through the at least one opening and at least momentarily held in the chamber responsive to the vacuum pressure.
